(19) United States
(12) Patent Application Publication
(10) Pub. No.: US 2022/0238238 A1
(43) Pub. Date: Jul. 28, 2022

(54) METHOD FOR NAVIGATING MEDICAL DATA

(71) Applicant: Novartis AG, Basel (CH)

(72) Inventors: Gregory LIGOZIO, Basel (CH); Luminita PRICOP, Basel (CH); Matthias KORMAKSSON, Basel (CH); Tingting ZHUANG, Basel (CH); Xuan ZHU, Basel (CH); David A. JAMES, Basel (CH); Effie POURNARA, Basel (CH); Jennifer FRUEH, Basel (CH)

(21) Appl. No.: 17/613,889

(22) PCT Filed: May 27, 2020
(86) PCT No.: PCT/EP2020/064719

§ 371 Date: Nov. 23, 2021

(30) Foreign Application Priority Data
Jun. 7, 2019 (EP) 19179139.1

(51) Int. Cl. G16H 50/70; G16H 20/90; G16H 50/20; G06N 3/08

(52) U.S. Cl. CPC G16H 50/70; G16H 20/90; G06F 3/04842; G06N 3/08; G16H 50/20

(57) ABSTRACT

The invention comprises a computer-implemented method comprising the steps of providing a medical dataset with data of a plurality of patients and pertaining to at least one disease, associating the data in the dataset with corresponding parts of the human body, to which the data pertains, displaying, on a display device, a graphical representation of the human body (1) or of a part of the human body, displaying, on the display device, a plurality of activatable buttons (2) associated with different parts of said graphical representation (1), receiving a user input activating one of the plurality of activatable buttons (2), and based on the activation, displaying, on the display device, a graphical representation of the data (4) associated with that part of the human body, to which the activatable button (2) activated by the user input pertains.

The invention relates to a computer-implemented method for navigating medical data, particularly large medical datasets with data of a plurality of patients.

Especially in the pharmaceutical industry, large medical surveys are conducted including a large number of patients. For the process of drug approval, clinical trials are carried out including thousands of patients. Clinical data, in the form of patient demographics, medical history, baseline disease characteristics, composite measurements of disease activity evaluations and patient reported outcomes (PROs) are collected at different time-points and over a long period of time, which may extend to 5 years of follow up. For instance, patients with psoriasis or psoriatic arthritis may be periodically examined on how the condition of their joints and/or their skin have developed since starting using a medication.

The resulting time-dependent data is usually available in the form of large tables, the so-called patient level data.

This immense amount of data is usually analyzed based on the Statistical Analysis Plan and used to develop the Clinical Study Report (CSR), which may sum up to 10,000 pages including the appendices. Only a very small proportion of these outputs are published in peer-reviewed journals and these are largely summary tables or statistical tests.

Such summaries, however, do not preserve the data in their anatomical and/or physiological integrity. For psoriatic arthritis, for instance, the question may arise whether the location of the swollen or tender joints may affect a treatment response. Such a question can apparently not be answered using the published summaries. Given the number of locations for possible joint pain (78 joints), the use of traditional patient level data for answering the question is also cumbersome if practicably possible at all.

It is therefore an object of the present invention to provide a computer-implemented method, which facilitates the efficient searching and evaluation of data of large medical datasets.

This object is achieved with a method according to claim 1. Preferred embodiments are specified in the dependent claims.

The method allows for efficiently searching specific data in their corresponding anatomical and/or physiological context. Moreover, it provides a framework for interactive exploration of medical datasets.

The medical dataset may comprise patient level data for a plurality of patients, particularly for more than 500 patients, particularly for more than 1,000 patients. For each patient, one or more medical data items may be stored in the medical dataset.

The medical dataset may particularly be a clinical dataset, in particular comprising data from one or more clinical trials.

The graphical representation of the human body or of the part of the human body may be a schematic representation or a realistic representation. The graphical representation may also include one or more anatomically correct representations of parts of the human body or an anatomically correct representation of the human body as a whole. The graphical representation of the human body or of a part thereof may be a two-dimensional (2D) or three-dimensional (3D) representation.

The graphical representation of the human body or of a part thereof may also be animated. In particular, the graphical representation of the human body or of a part thereof may change depending on the disease progression and/or the displayed graphical representation of the data.

The activatable buttons are virtual buttons displayed on the display device. The form of the activatable buttons is not particularly restricted. According to one embodiment, the color and/or form of the activatable buttons can change when activated. In this way, the user can easily identify the activated button.

The activatable buttons may be arranged respectively overlapping with or adjacent to the part of said graphical representation representing a certain part of the human body to which the activatable button pertains. In other words, the activatable buttons may be associated via the graphical representation to certain elements of the human body. For instance, an activatable button may pertain to a hand or foot of the human body, while another activatable button pertains to the head or shoulder. When the graphical representation provides further details or is a representation of a part of the human body, the activatable buttons may also pertain to smaller scale elements of the human body, such as joints, organs or blood vessels.

The activatable buttons may particularly be anchored to the respective parts of said graphical representation. The activatable buttons may be displayed such that they are visible on the display when the respective part of the graphical representation is visible. The activatable buttons may be re-displayed at an appropriate position if after a user input, for instance a zoom operation or a relocation of another window overlapping the respective button, said button would no longer be visible in its previous location.

The user may activate the activatable button in any suitable way, for instance, using a touch sensitive display or a mouse.

The method may further comprise displaying a context menu in response to the activation of an activatable button, the context menu offering a set of choices for determining the graphical representation of the data and/or for choosing a subset of the data associated with the respective part of the human body. In this way, the data used for the graphical representation of the data or details of said graphical representation may be further narrowed. For instance, the medical datasets may comprise different measures for quantifying a certain symptom. Using the context menu, one of said measures may be selected, so that only data within the medical dataset pertaining to said measure and to the respective part of the human body will be taken into account for the graphical representation of the data.

The graphical representation of the data may comprise one or more charts or graphs. For instance, the graphical representation may comprise a pie chart, a bar chart, a box plot, a scatter plot or a dot plot. The graphical representation of the data may alternatively or additionally comprise one or more images of one or more patients and/or one or more realistic representations of one or of an average patient. For instance, the images may show parts of the skin of the patients and/or lesions of realistic representations may be shown.

The graphical representation of the data may particularly indicate the temporal disease progression, for instance via one or more charts, graphs and/or tables indicating the temporal disease progression.

The term “realistic representations” refers to image data obtained from photos of one or more patients, but being different from the photos as such. The one or more images may also comprise one or more anatomical images such as X-ray images, MRIs, CT scans and/or angiograms.

Via the context menu mentioned above, the user may select a particular chart or graph used for the graphical representation of the data.

The medical dataset may comprise time-dependent data, wherein the graphical representation of the data comprises representations of different temporal snapshots of that data. In this way, the time evolution of symptoms of the at least one disease and/or a response of the one or more patients to a certain medication may be evaluated. For instance, a series of charts or graphs may be used as representations of the different temporal snapshots.

The method may further comprise displaying a temporal control element, such as a time line, for selecting a point in time to which the data for the graphical representation of the data shall pertain. The method may further comprise receiving a user input via the temporal control element and displaying the graphical representation of the data pertaining to the selected point in time. For instance, a slider element may be provided, which may be shifted along the time line to choose a point in time. In this way, the temporal disease progression can be efficiently explored by a user.

The graphical representation of the human body or of the part of the human body may be an interactive representation, in particular scalable and/or rotatable. In this way, it is possible to explore the medical dataset with respect to different parts of the human body. The graphical representation of the human body or of a part thereof may present a different level of detail depending on a zoom level. For instance, at a first zoom level the graphical representation of the human body may only show a schematic illustration of an element of the body, such as a hand. When zooming in, this graphical representation may change to a more detailed representation, particularly an anatomically correct representation, of the element of the human body, such as a hand comprising representations for the individual fingers and joints. Additional activatable buttons may be displayed for additional elements of the graphical representation, becoming visible after a zoom and/or rotation. Activatable buttons may also be removed from display if the corresponding part of the graphical representation is no longer visible, for instance after a zoom or rotation.

The medical dataset may pertain to more than one disease, wherein the method further comprises:

providing an interface allowing the user to select a disease; and

restricting the graphical representation of the data associated with the part of the human body, to which the activatable button activated by the user input pertains, to data pertaining to the selected disease. In this way, the medical dataset may be explored for different diseases. The method may in this case further comprise associating the data in the dataset with different diseases, to which the data pertains.

Alternatively or additionally, the medical dataset may pertain to more than one medication used for treating the at least one disease, wherein the method further comprises:

providing an interface allowing the user to select a medication; and

restricting the graphical representation of the data associated with the part of the human body to which the activatable button activated by the user input pertains, to data pertaining to the selected medication. In this way, the medical dataset may be explored with regard to different medications. This may aid the user in selecting the most appropriate medication. In this case, the method may further comprise associating the data in the dataset with the corresponding medication, to which the data pertains.

The graphical representation of the human body or of a part thereof, when animated, may change depending on the selected disease and/or medication. For instance, the graphical representation of the human body or of a part thereof may reflect the disease or the temporal progression thereof and/or the impact of the selected medication on the disease or the temporal progression thereof.

Associating the data in the dataset with corresponding parts of the human body, to which the data pertains, may comprise providing for the data of each patient at least one matrix, the elements of the matrix being each associated with a predetermined part of the human body and entering at least a part of the data or a quantity derived from said data into the at least one matrix. The matrix may also be an n×1 matrix, which is referred to herein as “vector”. There may be more than one matrix per patient, each matrix relating to different regions of the human body. For instance, one matrix may refer to the joints of the human body while a second matrix refers to different inner organs. Each matrix may be associated with a predetermined point in time. A temporal evolution of the patient level data may, thus, translate in a series of matrices, each corresponding to a different point in time.

Associating the data in the dataset with corresponding parts of the human body, to which the data pertains, may further comprise receiving and/or determining anatomical scores for each patient and storing them as elements in the at least one matrix. The anatomical scores, thus, may correspond to data in the medical dataset or to quantities derived from said data.

Anatomical scores are defined as clinical assessments or evaluations performed on specific anatomical locations. Joint scores (swelling, tenderness) are one example for anatomical scores. For instance, since 78 joints are monitored in psoriatric arthritis, the resulting matrix may be a 78×1 matrix, i.e. a vector with 78 entries.

Other anatomical scores are conceivable as well, e.g. enthesitis scores on entheses (locations where tendons ligaments attach to a bone), psoriatic skin scores (erythema on the head, trunk, etc.), dactylitis scores (for fingers, toes), and/or bone erosion and joint space narrowing scores (e.g. extracted from X-rays).

In other diseases anatomical scores may relate to edema (excess fluid) on the retina in patients with age-related macular edema (ADM) or diabetic retinopathy, blood velocity (e.g. measured by Doppler echocardiography) at various heart valves (location) and/or wall thickness in cardiomyopathy among heart failure patients. Anatomical scores may also relate to T2 lesions in the brain of Multiple sclerosis (MS) patients.

Associating the data in the dataset with corresponding parts of the human body, to which the data pertains, may further comprise determining anatomical scores from the patient level data of at least one patient using a machine learning technique and/or an artificial neural network, particularly a deep neural network. The anatomical scores may particularly be determined based on medical images such as X-Ray images, computed tomography (CT) scans and/or Magnetic resonance imaging (MRI).

Associating the data in the dataset with corresponding parts of the human body, to which the data pertains, may further comprise determining statistical quantities of the medical data based on the at least one matrix for each patient. Based on a subset of patients less than all patients of the dataset or based on all patients, statistical quantities, such as means, correlations, quantiles, and/or clustering, may be determined. Thus, the statistical quantities can be derived from full patient-level data.

For instance, for two patients with joint scores x1 and x2 the sum x1+x2, or the difference x1−x2, may be computed by corresponding matrix or vector operations. The result is again a matrix or vector.

The statistical quantities may be used for determining the graphical representation of the data associated with the part of the human body. For instance, joint scores of a corresponding mean vector may be displayed in a color-coded form overlaid the respective joint positions in a schematic representation of the human body.

The invention further provides a data processing apparatus comprising a processor adapted to perform the steps of the above-described method.

The invention further provides a computer program product comprising instructions, which, when the program is executed by a computer, cause the computer to carry out the steps of the above-described method.

The invention further provides a computer readable storage medium comprising instructions, which, when executed by a computer, cause the computer to carry out the steps of the above described method.

The steps of structuring the medical dataset as described above in relation to the step of associating the data in the dataset with corresponding parts of the human body, to which the data pertains, may also be embodied without the other steps of the method of claim 1.

In other words, the invention also provides a computer-implemented method for structuring a medical dataset with data of a plurality of patients and pertaining to at least one disease, comprising providing for the data of each patient at least one matrix, the elements of the matrix being each associated with a predetermined part of the human body and entering at least a part of the data or a quantity derived from said data into the at least one matrix.

As noted above, the matrix may also be an n×1 matrix, which is referred to herein as “vector”. There may be more than one matrix per patient, each matrix relating to different regions of the human body. For instance, one matrix may refer to the joints of the human body while a second matrix refers to different inner organs. Each matrix may be associated with a predetermined point in time. A temporal evolution of the patient level data may, thus, translate in a series of matrices, each corresponding to a different point in time.

Structuring the medical dataset may further comprise receiving and/or determining anatomical scores for each patient and storing them as elements in the at least one matrix. The anatomical scores, thus, may correspond to data in the medical dataset or to quantities derived from said data.

Anatomical scores are defined as clinical assessments or evaluations performed on specific anatomical locations. Joint scores (swelling, tenderness) are one example for anatomical scores. Since there are 78 joints in the human body, the resulting matrix may be a 78×1 matrix, i.e. a vector with 78 entries.

Other anatomical scores are conceivable as well, e.g. enthesitis scores on entheses (locations where tendons ligaments attach to a bone), psoriatic skin scores (erythema on the head, trunk, etc.), dactylitis scores (for fingers, toes), and/or bone erosion and joint space narrowing scores (e.g. extracted from X-rays).

In other diseases anatomical scores may relate to edema (excess fluid) on the retina in patients with age-related macular edema (ADM) or diabetic retinopathy, blood velocity (e.g. measured by Doppler echocardiography) at various heart valves (location) and/or wall thickness in cardiomyopathy among heart failure patients. Anatomical scores may also relate to T2 lesions in the brain of Multiple sclerosis (MS) patients.

Structuring the medical dataset may further comprise determining anatomical scores from the patient level data of at least one patient using a machine learning technique and/or an artificial neural network, particularly a deep neural network. The anatomical scores may particularly be determined based on medical images such as X-Ray images, computed tomography (CT) scans and/or Magnetic resonance imaging (MRI).

Structuring the medical dataset may further comprise determining statistical quantities of the medical data based on the at least one matrix for each patient. Based on a subset of patients less than all patients of the dataset or based on all patients, statistical quantities, such as means, correlations, quantiles, and/or clustering, may be determined. Thus, the statistical quantities can be derived from full patient-level data.

For instance, for two patients with joint scores x1 and x2 the sum x1+x2, or the difference x1−x2, may be computed by corresponding matrix or vector operations. The result is again a matrix or vector.

The invention further provides a data processing apparatus comprising a processor adapted to perform the steps of the above-described method.

The invention further provides a computer program product comprising instructions, which, when the program is executed by a computer, cause the computer to carry out the steps of the above-described method.

The invention further provides a computer readable storage medium comprising instructions, which, when executed by a computer, cause the computer to carry out the steps of the above described method.

The invention further provides a computer-implemented method for identifying patient phenotypes in a medical dataset with data of a plurality of patients and pertaining to at least one disease. A phenotype is defined as a composite of observed clinical characteristics. The method may comprise identifying clusters of patients where the probability of a symptom is homogeneous within each cluster. In other words, each cluster may comprise patients associated with a predetermined phenotype. The clusters may be identified using a machine learning technique and/or an artificial neural network, particularly a deep neural network.

Identifying clusters of patients may particularly comprise applying a hierarchical clustering algorithm, for instance Ward's method, to a part of the medical dataset or to the complete dataset.

The method may further comprise applying a mixture model to clinical endpoints in the medical dataset.

The method may be used in combination with any of the above-described method. For instance, the identified clusters may be used for determining the graphical representation of the data. The above-mentioned matrices may be used for identifying the clusters of patients. The medical dataset may comprise one or more of the above-identified features.

The method may be used in therapeutics, prognosis and clinical decision making and may be used in personalized medicine. Particularly, the clustering is based on the assumption that two patients share a cluster only if they have similar probability of presence of symptom across all clinical variables. Consequently, if a patient can be assigned to a predetermined cluster, a personalized treatment based on a predetermined optimized treatment for the cluster is possible.

Advantageous embodiments will now be described in combination with the enclosed figures.

In the figures,

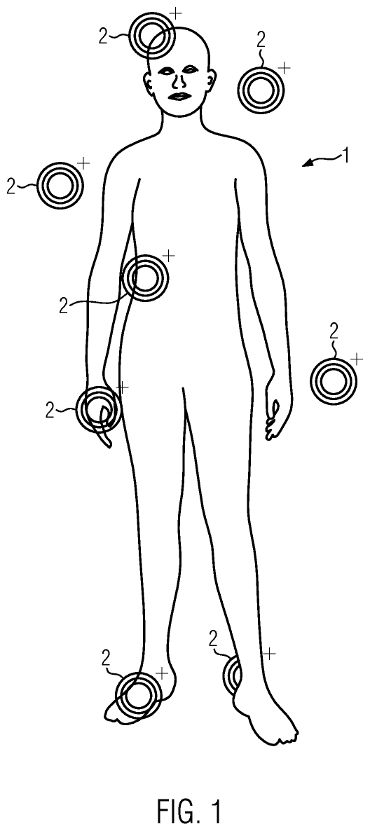

FIG. 1 illustrates an exemplary graphical representation of the human body;

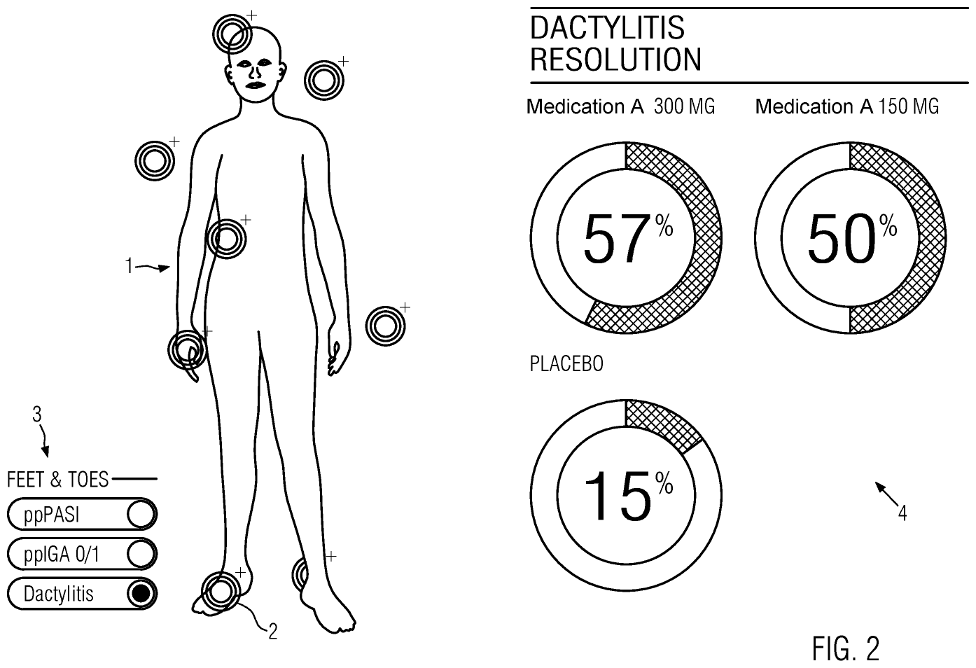

FIG. 2 illustrates an exemplary view for navigating medical data;

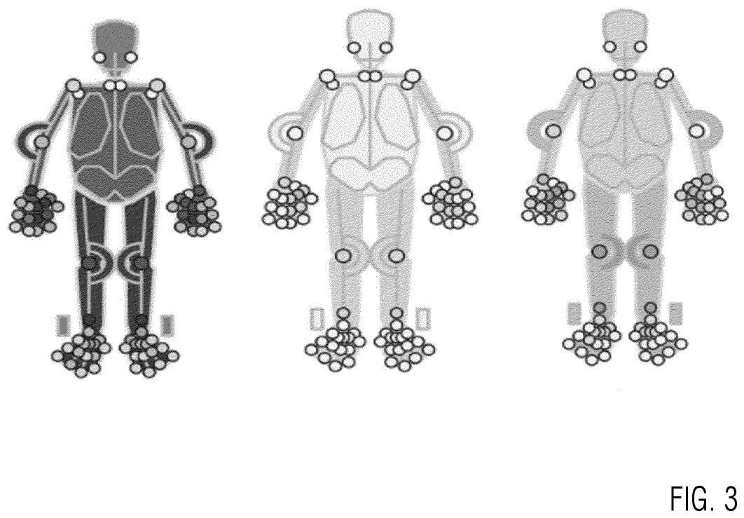

FIG. 3 illustrates an exemplary graphical representation of the data;

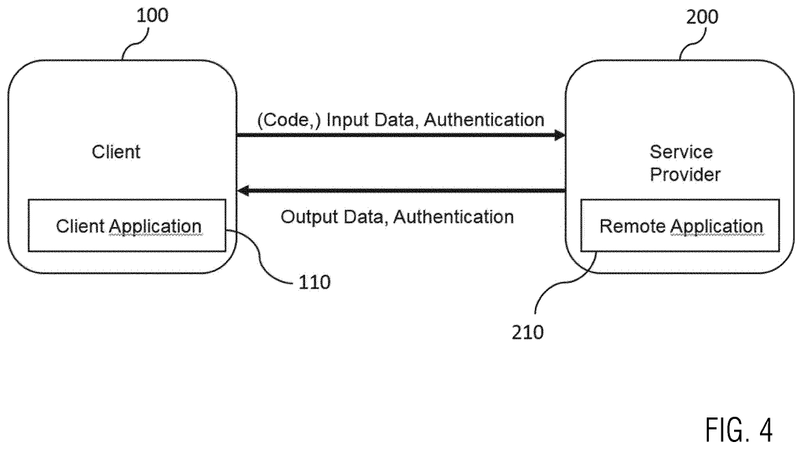

FIG. 4 illustrates an exemplary client-server system usable to implement the invention;

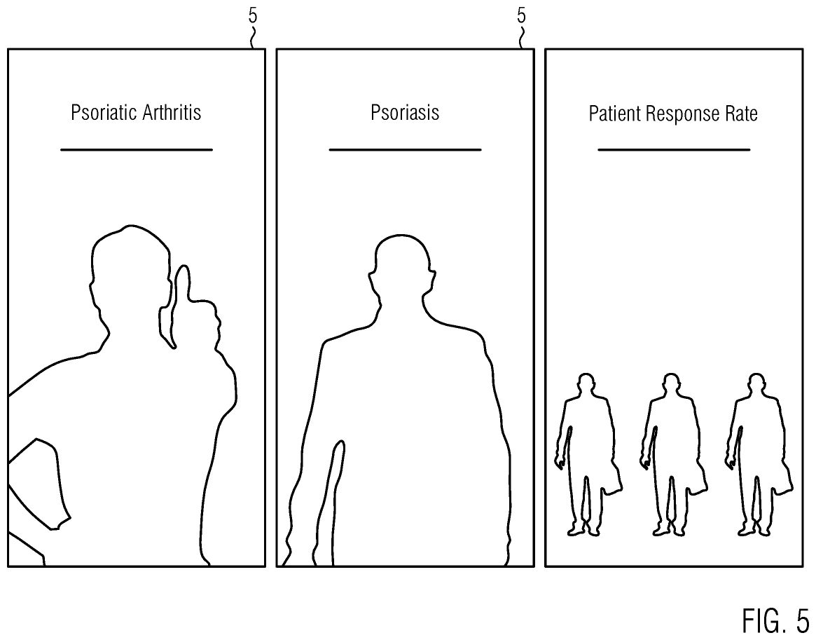

FIG. 5 illustrates an exemplary interface allowing a user to select a disease; and

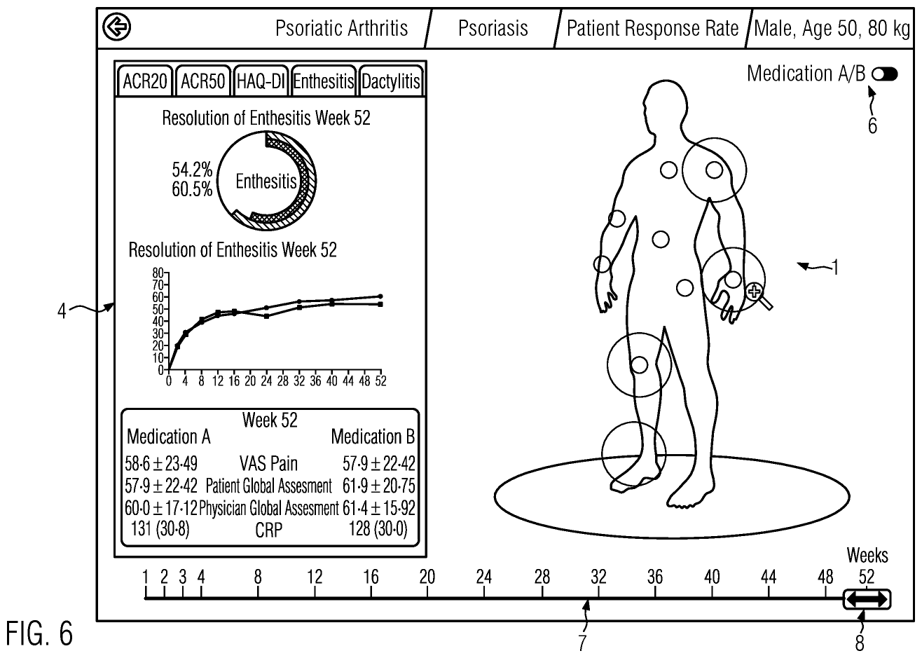

FIG. 6 illustrates a further exemplary view for navigating medical data.

In FIG. 1 an exemplary graphical representation of the human body 1 is shown. The representation 1 is a 3D-representation, which can be viewed from any direction upon a corresponding user input. The user can also zoom in into desired regions of the representation 1. In other words, the graphical representation 1 is an interactive representation, in particular a rotatable and scalable representation. The graphical representation 1 may also be referred to as “virtual patient”.

Associated with certain elements of the graphical representation 1 are activatable buttons 2. For instance, an activatable button 2 is associated with the head, another one with the hand and a third one with a foot. The activatable buttons 2 are anchored to the respective parts of the representation 1 and, thus, to the respective part of the human body. Consequently, when the graphical representation 1 is changed, for instance rotated and/or scaled, the activatable buttons 2 remain displayed as overlapping with or adjacent to the respective parts of the representation 1, as long as these parts are still discernible.

Upon activation of one of the activatable buttons 2, the button 2 may change its color and/or form. In this way, the user can easily recognize the activated button and, thus, the pertinent part of the human body.

As further described below, a context menu may be displayed in response to the activation of the activatable button 2. The availability of the context menu is indicated by the symbol “+” adjacent to each activatable button 2.

FIG. 2 illustrates an exemplary view after activating an activatable button 2 associated with feet and toes. A context menu 3 is displayed offering a set of—in this example three—choices.

Underlying this view is a medical dataset with data of a plurality of patients and pertaining to at least one disease, particularly psoriatic arthritis. The data in the dataset is associated with corresponding parts of the human body, to which the data pertains. In response to the activation of an activatable button 2 a graphical representation of the data 4 associated with that part of the human body, to which the activatable button 2 activated by the user input pertains, is displayed. The context menu 3 may be further used for determining the graphical representation of the data 4 and/or for choosing a subset of the data associated with the respective part of the human body. In the illustrated example, the context menu 3 allows choosing a subset of the data relating to a particular symptom, e.g. dactylitis (referring to the sausage-like swelling of the toes that can be present with slight redness and deformity).

The data in the dataset may be structured in the form of at least one matrix. In the matrix, dactylitis scores for the fingers/toes may be included. Each element of the matrix may pertain to another finger/toe. The matrix may pertain to a single patient or to an average patient. In other words, the matrix may comprise average scores.

A further non-illustrated context menu may be provided for choosing one or more diseases, one or more treatments and/or one or more data sources. A non-illustrated temporal control element may be provided, for instance in form of a time line, to choose one or more points in time to which the graphical representation of the data 4 shall pertain.

In the example illustrated in FIG. 2, the graphical representation of the data 4 comprises three doughnut charts, one for a treatment with a medication with a dose of 300 mg of a given substance, one for a treatment with a medication with a dose of 150 mg of the same substance and one for treatment with a placebo. The percentage indicates the dactylitis resolution for the treatment. Such a graphical representation of the data 4 allows the medical practitioner to easily and efficiently choose the appropriate treatment or allows the patients to estimate the prospect of healing.

Other charts are possible as well. Particularly, the graphical representation of the data 4 may illustrate different temporal snapshots of a temporal evolution of a feature of the underlying data.

The graphical representation of the data 4 may also include one or more graphical representations of the human body or of parts thereof. For instance, a color-coding may be used to indicate the severity of symptoms, for instance joint pain for different joints. Such a graphical representation of the data is illustrated in FIG. 3. Three temporal snapshots illustrate the temporal evolution of swollen or tender joints.

The dataset may also include one or more photos, for instance of the skin of patients. The graphical representation of the data 4 may display low-resolution versions of the high-resolution photos of the dataset, for instance of more than one patient for a certain point in time during treatment, or of one patient for more than one point in time during treatment. Upon selecting a photo via a user input, the high-resolution photo may be retrieved and displayed. In this way, an efficient search and retrieval of medical images is possible.

Instead of raw medical images also realistic representations may be provided and/or one or more anatomical images such as X-ray images, MRIs, CT scans and/or angiograms.

As noted above, a context menu 3 may be used to choose one or more diseases to which the data should pertain. FIG. 5 illustrates an alternative interface to choose one or more such diseases. Particularly, a selection screen may be displayed, comprising two or more selectable icons 5, associated with different diseases, in this example with Psoriatic Arthritis and Psoriasis, respectively. Upon selection of one of the selectable icons 5, the graphical representation may be restricted to data pertaining to the selected disease. In addition, the available activatable buttons 2 may depend on the selected disease.

FIG. 6 shows another exemplary view for navigating medical data. As in the embodiment of FIG. 2, a graphical representation of the human body 1 is displayed next to the graphical representation of the data 4. Additionally, an interface 6 allowing the user to select a medication is provided. The interface 6 is a virtual switch in this example, which allows switching between medication A and medication B, respectively. Depending on the state of the virtual switch, the graphical representation of the data 4 may be restricted to data pertaining to the selected medication.

Furthermore, a temporal control element is provided in the example of FIG. 6, particularly in form of a time line 7. To choose a point in time to which the graphical representation of the data 4 shall pertain, a slider element 8 is provided, which may be shifted along the time line 7 to choose a point in time.

The graphical representation of the human body 1 may be animated. For instance, it may perform movements to create a realistic impression of a human being. In particular, the graphical representation of the human body 1 may change depending on the disease progression and/or the displayed graphical representation of the data. For skin diseases, for instance, the virtual skin of the graphical representation of the human body 1 may represent the medical condition of the skin at a given temporal snapshot.

The described method allows navigating large medical datasets, such as those originating from a clinical trial, for instance, in a very efficient and illustrative manner. It is then possible, for example, to view the medical conditions and their evolution for specific parts of the human body, depending, for instance, on different medication or different application rates. This may help a medical practitioner in choosing the appropriate treatment, or the patient in estimating the further course of disease.

The invention may be embodied in a client-server architecture. Alternatively, the invention may be embodied entirely on a single computing device, i.e. as a stand-alone solution. In the latter case, the single computing device may comprise one or more of the features described below for a client.

A general client-server architecture for remote services is schematically illustrated in FIG. 4. The configuration includes one or more clients 100 that communicate with one or more servers 200. A client may be implemented in a computing device or client device as described below. A server may be implemented in a server device provided by a service provider and/or a cloud provider. The present disclosure is, however, not limited to these specific implementations but may be applied to any configuration wherein a local client (device) requests a service from a remote server (device) that is provided to the client by the server. Alternatively, as mentioned above, the invention can be implemented in the local client alone.

It is understood that the service may be provided by more than a single server or server device, but may itself rely on a distributed system architecture. The server may include for instance, a web server as a front end, an application server and a data base server. For simplicity, the remote entity providing the service, whether a single server or server device, a distributed or micro service based system, or the like will be referred to in the following as a service provider. Furthermore, the client is not limited to a single client or client device but may itself include a distributed system. The client may further act as a server itself in a distributed environment, for instance as an intermediary server. The term client is used herein to include any of the above-mentioned architectures and merely indicates that entity 100, e.g. a client device, receives a service from a remote entity 200, e.g. a server device. With regard to other aspects than the provision and reception of the remote service, the client 100 and the server 200 may even switch roles.

The client 100 and the service provider 200 may be operatively connected to one or more respective client data stores and server data stores (not shown) that can be employed to store information local to the respective client 100 and server 200, such as application code, application data, input data, output data, authentication data, and the like. The medical database may be stored, for instance, in a client data store or a server data store.

The client 100 and the service provider 200 may communicate information between each other using a communication framework as indicated by the arrows. The information may include authentication information such as keys and/or signatures for establishing a secure communication channel, one or more applications, e.g. as code or binaries, input data and/or configuration data for execution of the remote application, output data of the remote application, and the like. The information may further include keys and/or signatures for software attestation as described below. Furthermore, applications may be provided as interpreted code to be executed by an interpreter.

In an IaaS (Infrastructure as a Service) architecture, the remote application may be provided by the client 100 and communicated to the service provider 200 via the communication channel before it is executed by the service provider. In this case, the remote service may include installing, e.g. compiling, or interpreting the application code received from the client, executing the received application as a remote application on the side of the service provider, and communicating the results of the execution back to the client 100. In an SaaS (Software as a Service) architecture, the remote application is provided by the service provider itself and the remote service includes executing the remote application, potentially on input data and/or configuration data received from the client 100, and communicating the results to the client.

The communications framework used for communications between the client 100 and the service provider 200 may implement any well-known communication techniques and protocols. The communications framework may be implemented as a packet-switched network (e.g., public networks such as the Internet, private networks such as an enterprise intranet, and so forth), a circuit-switched network (e.g., the public switched telephone network), or a combination of a packet-switched network and a circuit-switched network (with suitable gateways and translators). The client-server architecture may include various common communications elements, such as a transmitter, receiver, transceiver, radio, network interface, baseband processor, antenna, amplifiers, filters, power supplies, and so forth. The embodiments, however, are not limited to these implementations.

The communications framework may implement various network interfaces arranged to accept, communicate, and connect to a communications network. A network interface may be regarded as a specialized form of an input/output interface. Network interfaces may employ connection protocols including without limitation direct connect, Ethernet (e.g., thick, thin, twisted pair 10/100/1000 Base T, and the like), token ring, wireless network interfaces, cellular network interfaces, IEEE 802.11a-x network interfaces, IEEE 802.16 network interfaces, IEEE 802.20 network interfaces, and the like. Further, multiple network interfaces may be used to engage with various communications network types. For example, multiple network interfaces may be employed to allow for the communication over broadcast, multicast, and unicast networks. Should processing requirements dictate a greater speed and capacity, distributed network controller architectures may similarly be employed to pool, load balance, and otherwise increase the communication bandwidth required by clients 100 and servers 200. A communications network may be any one and the combination of wired and/or wireless networks including without limitation a direct interconnection, a secured custom connection, a private network (e.g., an enterprise intranet), a public network (e.g., the Internet), a Personal Area Network (PAN), a Local Area Network (LAN), a Metropolitan Area Network (MAN), an Operating Missions as Nodes on the Internet (OMNI), a Wide Area Network (WAN), a wireless network, a cellular network, and other communications networks.

As mentioned above, the client 100 and the server 200 may each include a device that may be any electronic device capable of receiving, processing, and sending information, e.g. through a communication component. Examples of a computing device or electronic device may include without limitation a client device, a personal digital assistant (PDA), a mobile computing device, a smart phone, a cellular telephone, ebook readers, a messaging device, a computer, a personal computer (PC), a desktop computer, a laptop computer, a notebook computer, a netbook computer, a handheld computer, a tablet computer, a server, a server array or server farm, a web server, a network server, an Internet server, a work station, a network appliance, a web appliance, a distributed computing system, a multiprocessor system, a processor-based system, consumer electronics, programmable consumer electronics, a game device, a television, a set top box, a wireless access point, a base station, a subscriber station, a mobile subscriber center, a radio network controller, a router, a hub, a gateway, a bridge, a switch, a machine, or a combination thereof. The embodiments are not limited in this context.

The device may execute processing operations or logic for the one or several applications such as the exemplary client application 110 and the remote application 210, for a communications component, the operating system, in particular a kernel of the operating system, and for other software elements using one or more processing components, i.e. processing circuitry. The processing components or processing circuitry may comprise various hardware elements such as devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate arrays (FPGA), memory units, logic gates, registers, semiconductor devices, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof.

The device may execute communications operations or logic for communications with other devices using one or more communications components. The communications components may implement any well-known communications techniques and protocols, such as techniques suitable for use with packet-switched networks (e.g., public networks such as the Internet, private networks such as an enterprise intranet, and so forth), circuit-switched networks (e.g., the public switched telephone network), or a combination of packet-switched networks and circuit-switched networks (with suitable gateways and translators). The communications component may include various types of standard communication elements, such as one or more communications interfaces, network interfaces, network interface cards (NIC), radios, wireless transmitters/receivers (transceivers), wired and/or wireless communication media, physical connectors, and so forth. By way of example, and not limitation, communication media include wired communications media and wireless communications media. Examples of wired communications media may include a wire, cable, metal leads, printed circuit boards (PCB), backplanes, switch fabrics, semiconductor material, twisted-pair wire, co-axial cable, fiber optics, a propagated signal, and so forth. Examples of wireless communications media may include acoustic, radio-frequency (RF) spectrum, infrared and other wireless media.

The device may communicate with other devices over communications media using communications signals as indicated in FIG. 1, via the one or more communications components. The other devices may be internal or external to the device, as desired for a given implementation.

The device may be implemented in the form of a distributed system that may distribute portions of the above-described structure and/or operations across multiple computing entities. Examples of a distributed system may include without limitation a client-server architecture, a 3-tier architecture, an N-tier architecture, a tightly-coupled or clustered architecture, a peer-to-peer architecture, a master-slave architecture, a shared database architecture, and other types of distributed systems. The embodiments are not limited in this context.

The client 100 and/or the server 200 may include a computing architecture as described in the following. In one embodiment, the computing architecture may comprise or be implemented as part of an electronic device. Examples of an electronic device may include those described above. The embodiments are not limited in this context.

As used in this application, the terms “apparatus”, “component”, “client”, “server”, “service provider”, and “software provider” are intended to refer to a computer-related entity, either hardware, a combination of hardware and software, software, or software in execution, examples of which are provided by the exemplary computing architecture described below. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer or distributed between two or more computers. Further, components may be communicatively coupled to each other by various types of communications media to coordinate operations. The coordination may involve the uni-directional or bi-directional exchange of information as required. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture may include various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by this computing architecture.

The computing architecture may comprise a processing unit, a system memory, and a system bus. The processing unit can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit.

The system bus provides an interface for system components including, but not limited to, the system memory to the processing unit. The system bus can be any of several types of bus structure that may further interconnect to a memory bus (with or without a memory controller), a peripheral bus, and a local bus using any of a variety of commercially available bus architectures. Interface adapters may connect to the system bus via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computing architecture may comprise or implement a computer-readable storage medium to store logic. Examples of a computer-readable storage medium may include any tangible media capable of storing electronic data, including volatile memory or non-volatile memory, removable or non-removable memory, erasable or non-erasable memory, writable or re-writable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD)) and any other type of storage media suitable for storing information. The system memory can include non-volatile memory and/or volatile memory. A basic input/output system (BIOS) can be stored in the non-volatile memory.

The computing architecture may include various types of computer-readable storage media in the form of one or more lower speed memory units, including an internal (or external) hard disk drive (HDD), a magnetic floppy disk drive (FDD) to read from or write to a removable magnetic disk, and an optical disk drive to read from or write to a removable optical disk (e.g., a CD-ROM, DVD, or Blu-ray). The HDD, FDD and optical disk drive can be connected to the system bus by a HDD interface, an FDD interface and an optical drive interface, respectively. The HDD interface for external drive implementations can include at least one or both of Universal Serial Bus (USB) and IEEE 1394 interface technologies.

The drives and associated computer-readable media provide volatile and/or nonvolatile storage of data, data structures, computer-executable instructions, and so forth. For example, a number of program modules can be stored in the drives and memory units, including an operating system, in particular a kernel of an operating system, one or more application programs, also called applications herein, such as the exemplary client application 110 and the exemplary remote application 210, other program modules, and program data. In one embodiment, the one or more application programs, other program modules, and program data can include, for example, the various applications and/or components to implement the disclosed embodiments.

A user can enter commands and information into the computing device through one or more wire/wireless input devices, for example, a keyboard and a pointing device, such as a mouse. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit through an input device interface that is coupled to the system bus, but can be connected by other interfaces such as a parallel port, IEEE 1394 serial port, a game port, a USB port, an IR interface, and so forth.

A display device may also be connected to the system bus via an interface, such as a video adaptor. The display device may be internal or external to the computing device. In addition to the display device, a computing device typically includes other peripheral output devices, such as speakers, printers, and so forth.

The computing device may operate in a networked environment using logical connections via wire and/or wireless communications to one or more remote computers, such as a remote device. The remote device can be a workstation, a server computer, a router, a personal computer, portable computer, microprocessor-based entertainment appliance, a peer device or other common network node, and typically includes many or all of the elements described relative to the computing architecture. The logical connections may include wire/wireless connectivity to a local area network (LAN) and/or larger networks, for example, a wide area network (WAN). Such LAN and WAN networking environments are commonplace in offices and companies, and facilitate enterprise-wide computer networks, such as intranets, all of which may connect to a global communications network, for example, the Internet.

When used in a LAN networking environment, the device is connected to the LAN through a wire and/or wireless communications network interface or adaptor. The adaptor can facilitate wire and/or wireless communications to the LAN, which may also include a wireless access point disposed thereon for communicating with the wireless functionality of the adaptor.

When used in a WAN networking environment, the device can include a modem, or is connected to a communications server on the WAN, or has other means for establishing communications over the WAN, such as by way of the Internet. The modem, which can be internal or external and a wire and/or wireless device, connects to the system bus via the input device interface. In a networked environment, program modules, or portions thereof, can be stored in a remote memory/storage device. It will be appreciated that the network connections are exemplary and other means of establishing a communications link between the devices can be used.

The client/server device is operable to communicate with wire and wireless devices or entities using the IEEE 802 family of standards, such as wireless devices operatively disposed in wireless communication (e.g., IEEE 802.11 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect devices to each other, to the Internet, and to wire networks (which use IEEE 802.3-related media and functions).

Although the previously discussed embodiments and examples of the present invention have been described separately, it is to be understood that some or all of the above-described features can also be combined in different ways. The above-discussed embodiments are not intended as limitations, but serve as examples, illustrating features and advantages of the invention.

1. A computer-implemented method comprising:
providing a medical dataset with data of a plurality of patients and pertaining to at least one disease;
associating the data in the dataset with corresponding parts of the human body, to which the data pertains;
displaying, on a display device, a graphical representation of the human body or of a part of the human body;
displaying, on the display device, a plurality of activatable buttons associated with different parts of said graphical representation;
receiving a user input activating one of the plurality of activatable buttons; and
based on the activation, displaying, on the display device, a graphical representation of the data associated with that part of the human body, to which the activatable button activated by the user input pertains.

2. The computer-implemented method according to claim 1, further comprising displaying a context menu in response to the activation of the activatable button, the context menu offering a set of choices for determining the graphical representation of the data and/or for choosing a subset of the data associated with the respective part of the human body.

3. The computer-implemented method according to claim 1, wherein the graphical representation of the data comprises one or more charts.

4. Computer-implemented method according to claim 1, wherein the medical dataset comprises time-dependent data and wherein the graphical representation of the data comprises representations of different temporal snapshots of the data, in particular, wherein a temporal control element is provided, particularly in form of a time line, to choose one or more temporal snapshots to which the graphical representation of the data shall pertain.

5. The computer-implemented method according to claim 1, wherein the graphical representation of the human body or of a part of the human body is an interactive representation, in particular scalable and/or rotatable.

6. The computer-implemented method according to claim 1, wherein the medical dataset pertains to more than one disease and wherein the method further comprises:
providing an interface allowing the user to select a disease; and
restricting the graphical representation of the data associated with the part of the human body, to which the activatable button activated by the user input pertains, to data pertaining to the selected disease.

7. The computer-implemented method according to claim 1, wherein the medical dataset pertains to more than one medication used for treating the at least one disease, and wherein the method further comprises:
providing an interface allowing the user to select a medication; and
restricting the graphical representation of the data associated with the part of the human body, to which the activatable button activated by the user input pertains, to data pertaining to the selected medication.

8. The computer-implemented method according to claim 1, wherein associating the data in the dataset with corresponding parts of the human body, to which the data pertains, comprises providing for the data of each patient at least one matrix, the elements of the matrix being each associated with a predetermined part of the human body and entering at least a part of the data or a quantity derived from said data into the at least one matrix.

9. The computer-implemented method according to claim 8, wherein associating the data in the dataset with corresponding parts of the human body, to which the data pertains, further comprises receiving and/or determining anatomical scores for each patient and storing them as elements in the at least one matrix.

10. The computer-implemented method according to claim 9, further comprising determining anatomical scores from the patient level data of at least one patient using a machine learning technique and/or an artificial neural network, particularly a deep neural network.

11. A data processing apparatus comprising:
a processor adapted to perform the steps of:
providing a medical dataset with data of a plurality of patients and pertaining to at least one disease;
associating the data in the dataset with corresponding parts of the human body, to which the data pertains;
displaying, on a display device, a graphical representation of the human body or of a part of the human body;
displaying, on the display device, a plurality of activatable buttons associated with different parts of said graphical representation;
receiving a user input activating one of the plurality of activatable buttons; and
based on the activation, displaying, on the display device, a graphical representation of the data associated with that part of the human body, to which the activatable button activated by the user input pertains.

12. The data processing apparatus according to claim 11, wherein the processor is further adapted to perform the step of displaying a context menu in response to the activation of the activatable button, the context menu offering a set of choices for determining the graphical representation of the data and/or for choosing a subset of the data associated with the respective part of the human body.

13. The data processing apparatus according to claim 12, wherein the graphical representation of the data comprises one or more charts.

14. The data processing apparatus according to claim 11, wherein the medical dataset comprises time-dependent data and wherein the graphical representation of the data comprises representations of different temporal snapshots of the data, in particular, wherein a temporal control element is provided, particularly in form of a time line, to choose one or more temporal snapshots to which the graphical representation of the data shall pertain.

15. The data processing apparatus according to claim 11, wherein the graphical representation of the human body or of a part of the human body is an interactive representation, in particular scalable and/or rotatable.

16. A computer-readable storage medium comprising instructions which, when executed by a computer, cause the computer to carry out the steps of:
providing a medical dataset with data of a plurality of patients and pertaining to at least one disease;
associating the data in the dataset with corresponding parts of the human body, to which the data pertains;
displaying, on a display device, a graphical representation of the human body or of a part of the human body;
displaying, on the display device, a plurality of activatable buttons associated with different parts of said graphical representation;
receiving a user input activating one of the plurality of activatable buttons; and
based on the activation, displaying, on the display device, a graphical representation of the data associated with that part of the human body, to which the activatable button activated by the user input pertains.

17. The computer-readable storage medium according to claim 16, wherein the medical dataset pertains to more than one disease and wherein the computer-readable storage medium comprises further instructions which, when executed by the computer, cause the computer to carry out the steps of:
providing an interface allowing the user to select a disease; and
restricting the graphical representation of the data associated with the part of the human body, to which the activatable button activated by the user input pertains, to data pertaining to the selected disease.

18. The computer-readable storage medium according to claim 16, wherein the medical dataset pertains to more than one medication used for treating the at least one disease, and wherein the computer-readable storage medium comprises further instructions which, when executed by the computer, cause the computer to carry out the steps of:
providing an interface allowing the user to select a medication; and
restricting the graphical representation of the data associated with the part of the human body, to which the activatable button activated by the user input pertains, to data pertaining to the selected medication.

19. The computer-readable storage medium according to claim 16, wherein associating the data in the dataset with corresponding parts of the human body, to which the data pertains, comprises providing for the data of each patient at least one matrix, the elements of the matrix being each associated with a predetermined part of the human body and entering at least a part of the data or a quantity derived from said data into the at least one matrix.

20. The computer-readable storage medium according to claim 19, wherein associating the data in the dataset with corresponding parts of the human body, to which the data pertains, further comprises receiving and/or determining anatomical scores for each patient and storing them as elements in the at least one matrix.
